SEQUENCE LISTING (18 sequences)

SEQ ID NO: 1 - protein, 5 aa, Staphylococcus aureus
  SITE 3: motif in CNA that anchors to the peptidoglycan in the cell wall, Xaa = any
KPXTG

SEQ ID NO: 2 - DNA, 31 bp, Artificial sequence
  primer for BA0871L
gaaggatccacagaattaaaaggtttaggtg

SEQ ID NO: 3 - DNA, 33 bp, Artificial sequence
  primer for BA0871RPQE
gaagtcgactcattctttcgaaattgctttacc

SEQ ID NO: 4 - DNA, 30 bp, Artificial sequence
  primer for BA5258L
gaaggatccaacagggaaacatataagatg

SEQ ID NO: 5 - DNA, 33 bp, Artificial sequence
  primer for BA5258RPQE
gaagtcgactcacagctctaatacagcagtttc

SEQ ID NO: 6 - DNA, 28 bp, Artificial sequence
  primer for CNAB5
gaagtcgacacaacatcaattagtggtg

SEQ ID NO: 7 - DNA, 30 bp, Artificial sequence
  primer for PSTCNA3
aacctgcagtacatagaactaagaatagcc

SEQ ID NO: 8 - DNA, 30 bp, Artificial sequence
  primer for BA5258RYX105
gaagtcgaccagctctaatacagcagtttc

SEQ ID NO: 9 - DNA, 30 bp, Artificial sequence
  primer for BA0871RYX015
gaagtcgacttctttcgaaattgctttacc

SEQ ID NO: 10 - DNA, 2910 bp, Bacillus anthracis
  nucleotide sequence encoding cell wall anchoring protein BA0871
atgaattttttgaggaaatcatttaatcaaaaaataaataaattaagtagtagttttatagtcgtgttactagtatgtatgaattttttaatccatttaccatataaagcagaggcagctacaacagaattaaaaggtttaggtgatgtatcttattacaatgcaatcatttttggagatcatagtgcaacgagtgcggatattgagggtgcgatggctattcaaaaaaatatgaatgcatcaagttatacagtcttagcggctgcaacgggagcaaataacttagcaggagcaacttgggtagatgaaggatatccatcattattactaggcggtcaatttacgaaagcgggagcaggacaagtaattattcaagatggaacagtggcgatgacaaaagacggagatccagatagtgcaatgaaaacgtcatatgaccgtatctcttataaagagcaagcggagattgatgctaagtttaaagaatttagaaaagacgttgatggtgtaattggggatgcaagtaaactacagactgataagccaaaacctaatatgagctttggaatcggtgaagatgtaaacaaccctaatatttatgtctcttcaggccaaacgggaaagaaagcatttgatgtaacagacgtttttcttccgaatgtagagaataaagactttattgttatttattcagatgcagaagaagtgagttttggtagcggtgcaattttgtatgatacaagaaatactgggatggcgacggatttaattaatacatctcaagcatacgaccctaattcatcttttactgagttagctagtaaggtaatttgggtatttcctaatgctacaaagataacaactaaaggttatggtgtagtaggtagtgtgtttgcacctaatgcagttgtagaaacaaaaggtggttctattaatggacaagcctatgttggaggattacaccaaagagacggttttgaggttcataactttaaatttaactggccaaagtggaaaaagccagcagctaaaaaaggaaatttacaaattaaaaaagttgatgaaaacaatgaaaatattgttttaaaagacgcaaaatttgatgttatagataaagagaataatgttgtagatacggttacaacaaacgaaaagggtattgcggaagttaaagatttaccatttggtgattattttgtaaaagaaattagtgcaccagaaggatatataaagattgatgcaccagtaaaagtaacaattgataatacaaacataattgaatttgttataaagaatacgaaaaaattagaaaatggtcaattaaaattattgaaaaaagatagtgaatctggtcaacttctaccaggtgcaaaatttgatgttatcgataaagatgggaaagttgtggaaacaattgttacagatgataaaggtgaagctttatcgaaacaacttccagttggaagctatacattaaaagaagtagaagcaccgaaaggatatgaattatcatctagttcagtttctgttgatgtagaggctaataaagtagtgactgtagatgtggtgaataaaaagatccccgaaaaagtaacaggtcaatttgaagtggtgaaagtagatgcaaatgataaaacgaaattgttatcaggtgcagaattcgaagtgtataaagatggcaaaaaggtagcagaactgaaaacaggtgagagtggaaaagtgatgtcaccgaaattaccgctaggtgaatacacagtgaaagaaacgaaagcaccagcgggctacaagctttcagataaagaatggaaagtaacaattcaaaacgagaaagaagtagtaaaagtagaggcagaaaacgaaaaaatcttaggttctctacaaattattaaaatggatgataaagatcaaacgaaacgcttagcaggcgcagaatttacattgaaagatgtgaaaggcaatgttgtaaaagaaggaattacaacagataagtctggaactgttaaagtagacggacttgtgccgggtgaatatacgttagaagaaacaaaagcgccagaaggttataaggcattagaagtaacaatcgaagtaaacgtagtagcaaacgaagtagtaaaacaagacgtgttgaatgaaaaagtgaaagaagaaattacagggcaattagaaattacaaaggtagatgctaatgatataaataaaaaattagcaggcgcagtgtttgaaatttggaaagacggaacaaaaatcgatacattaacatcagatgaaaatggtaaagcaatttcgaaagaactggatccaggagattatattttaaaggaagttcaagcgccagaaggttatgagttatctgataaggaaatcgaatttacgatttctaatcaaaaatttgaagttgtaaaacttcaaattacaaataaaaaagaaacaagcaaaggtccagagaatccaggcgaagaaacagaaaagccaggtgaagaaacagaaaagccgggtgaagaaacagaaaagccaggcgaagaaacagaaaagccaggtgaagaaacagaaaagccaggtgaagaaacagaaaagccaggcgaagaaacagaaaagccaggtgaagaaacagaaaagccaggtgaagaaacagaaaagccaggcgaagaaacagaaaagccaggtgaagaaacagaaaagccaggcggagagacagaaaaaccgggtaaagaaacagagaaaccaggtgaaggaatggaaaacccagataaagaaaaagaaaatcctactttaccagaaaaaggacaaggtacctctcatgctcaacagcttccagccacaggacatgatatgaattatcttccattcattgggtttgctcttgttttattagggatacgcttaagatttatgactaaaaataactaa

SEQ ID NO: 11 - DNA, 1884 bp, Bacillus anthracis
  nucleotide sequence encoding cell wall anchoring protein BA5258
atgaaacgaaagatggtaggaaaatggtttagttttttaagtgctcttattattttattaggaattgcaatgccacaagtaaaggcagaagtaatgaacagggaaacatataagatggattggagctatagtaattcgaaacagcgtgaaataaagaccgaaataattaaaacggcttcggggagtattgcgtattgtttaacgcctgatttacgttctcccaatggagaagatttgcctgaaatggggaaaacatctgatgccgtatatcgtgttttattaaatggatatcctcaaaaaggcccatctgaattaggtgtagcgacaacggaagaagcgcattatgctacacaattagcagtatggattgcagcaaatgaattaacggaagaagatttagttgcaaaaaatgaacgtgtacataatcttatgaagcgtttagtagaggcttcaaagaaagaaactggatcacaagatgtattctttaaagttaatcctgtagattcacaaactgctacacaaaatggtgattatttagaaacaggattttatgcagtgcaaacaaatgcggtttctggctcttatacaattcttcctgaaaatgctcctaaagggcttcgaattgtaaatgaaaatggtgaagagaaaagtacgttatcaattaacgaaaaattcaaaattttacttccgaaagatacgagtagtggtaactttaaaatgaaagtgaagtctactttaacaaatttacaagcaatagcttttaaaggatcagaaaaagttcaaaatacaacggtattgttacaaagaaatagtgagaaaatcagtacagatttagttgtaaattgggaatcagtaggctctttaaagattatgaaattaggagaaaaaaaggaagtattaaaaggagcggtgtttgaagtttcgaatgaaaactttaaacaaaatgttacgactagtgataaggggattgcggagttaggtaatcttccaatcggtatatatagcgtcaaagaaattcaagcgccagctggatatgtgttagacagaagtgttaaaaaaattgaagtgaaaactggtgaaactgctgtattagagctgaaaaatgaaaatgtaaaaggtgaattagaaataacaaaagtagatgtagcggatggaaatacgaaactgccgaatgcagaatttacaatctacaatgaacaagggaaagaagtagtaaaaggaaaaacggatgaaaaaggtgtagcgaaattcaaactaccatacggaaaatacacgtataaagaaacaatagcaccaaatggatatgtaataaatgaagagacattcgcatttgaaataaaagagaatggagaaattattaaacatatcgttcaagataagaaggtagaaggtgagttagaaataacgaaagtagatgtagcagatgggaatacgaaattaccaaatgcagagtttacaatctacaacgaacaagggaaagaagtagtaaaaggaaaaacaaatgagcaaggtatagcgaaattcaaactaccatacggaaagtacacgtataaagaaacgatagcgccaaatggatatgtaataaatgaagagaagttcggatttgaaataaaagagaatggagaaattattaaacatattgtgaaaaataagaaagaagaaaaggcatcgtttccttctaagccaaataagccaacaccgaatggagaagtgaaaccttctgctgatgtgaagccaatgcaacaacctagtactcataatgaagtgcgtttaccagctactggcggtgtcagcaatcaatttacgtctttattcgttttaggaattagttttattattgcgggtgcttatgtgttaagaatgaaaaatagaaaagaaatgtag

SEQ ID NO: 12 - protein, 969 aa, Bacillus anthracis
  amino acid sequence of cell wall anchoring protein BA0871
MNFLRKSFNQKINKLSSSFIVVLLVCMNFLIHLPYKAEAATTELKGLGDVSYYNAIIFGDHSATSADIEGAMAIQKNMNASSYTVLAAATGANNLAGATWVDEGYPSLLLGGQFTKAGAGQVIIQDGTVAMTKDGDPDSAMKTSYDRISYKEQAEIDAKFKEFRKDVDGVIGDASKLQTDKPKPNMSFGIGEDVNNPNIYVSSGQTGKKAFDVTDVFLPNVENKDFIVIYSDAEEVSFGSGAILYDTRNTGMATDLINTSQAYDPNSSFTELASKVIWVFPNATKITTKGYGVVGSVFAPNAVVETKGGSINGQAYVGGLHQRDGFEVHNFKFNWPKWKKPAAKKGNLQIKKVDENNENIVLKDAKFDVIDKENNVVDTVTTNEKGIAEVKDLPFGDYFVKEISAPEGYIKIDAPVKVTIDNTNIIEFVIKNTKKLENGQLKLLKKDSESGQLLPGAKFDVIDKDGKVVETIVTDDKGEALSKQLPVGSYTLKEVEAPKGYELSSSSVSVDVEANKVVTVDVVNKKIPEKVTGQFEVVKVDANDKTKLLSGAEFEVYKDGKKVAELKTGESGKVMSPKLPLGEYTVKETKAPAGYKLSDKEWKVTIQNEKEVVKVEAENEKILGSLQIIKMDDKDQTKRLAGAEFTLKDVKGNVVKEGITTDKSGTVKVDGLVPGEYTLEETKAPEGYKALEVTIEVNVVANEVVKQDVLNEKVKEEITGQLEITKVDANDINKKLAGAVFEIWKDGTKIDTLTSDENGKAISKELDPGDYILKEVQAPEGYELSDKEIEFTISNQKFEVVKLQITNKKETSKGPENPGEETEKPGEETEKPGEETEKPGEETEKPGEETEKPGEETEKPGEETEKPGEETEKPGEETEKPGEETEKPGEETEKPGGETEKPGKETEKPGEGMENPDKEKENPTLPEKGQGTSHAQQLPATGHDMNYLPFIGFALVLLGIRLRFMTKNN

SEQ ID NO: 13 - protein, 627 aa, Bacillus anthracis
  amino acid sequence of cell wall anchoring protein BA5258
MKRKMVGKWFSFLSALIILLGIAMPQVKAEVMNRETYKMDWSYSNSKQREIKTEIIKTASGSIAYCLTPDLRSPNGEDLPEMGKTSDAVYRVLLNGYPQKGPSELGVATTEEAHYATQLAVWIAANELTEEDLVAKNERVHNLMKRLVEASKKETGSQDVFFKVNPVDSQTATQNGDYLETGFYAVQTNAVSGSYTILPENAPKGLRIVNENGEEKSTLSINEKFKILLPKDTSSGNFKMKVKSTLTNLQAIAFKGSEKVQNTTVLLQRNSEKISTDLVVNWESVGSLKIMKLGEKKEVLKGAVFEVSNENFKQNVTTSDKGIAELGNLPIGIYSVKEIQAPAGYVLDRSVKKIEVKTGETAVLELKNENVKGELEITKVDVADGNTKLPNAEFTIYNEQGKEVVKGKTDEKGVAKFKLPYGKYTYKETIAPNGYVINEETFAFEIKENGEIIKHIVQDKKVEGELEITKVDVADGNTKLPNAEFTIYNEQGKEVVKGKTNEQGIAKFKLPYGKYTYKETIAPNGYVINEEKFGFEIKENGEIIKHIVKNKKEEKASFPSKPNKPTPNGEVKPSADVKPMQQPSTHNEVRLPATGGVSNQFTSLFVLGISFIIAGAYVLRMKNRKEM

SEQ ID NO: 14 - protein, 5 aa, Bacillus anthracis
  amino acid sequence of cell wall anchoring protein motif in BA0871 and BA5258
KPATG

SEQ ID NO: 15 - DNA, 2172 bp, Bacillus anthracis
  nucleotide sequence of collagen-binding region A of BA0871
acagaattaaaaggtttaggtgatgtatcttattacaatgcaatcatttttggagatcatagtgcaacgagtgcggatattgagggtgcgatggctattcaaaaaaatatgaatgcatcaagttatacagtcttagcggctgcaacgggagcaaataacttagcaggagcaacttgggtagatgaaggatatccatcattattactaggcggtcaatttacgaaagcgggagcaggacaagtaattattcaagatggaacagtggcgatgacaaaagacggagatccagatagtgcaatgaaaacgtcatatgaccgtatctcttataaagagcaagcggagattgatgctaagtttaaagaatttagaaaagacgttgatggtgtaattggggatgcaagtaaactacagactgataagccaaaacctaatatgagctttggaatcggtgaagatgtaaacaaccctaatatttatgtctcttcaggccaaacgggaaagaaagcatttgatgtaacagacgtttttcttccgaatgtagagaataaagactttattgttatttattcagatgcagaagaagtgagttttggtagcggtgcaattttgtatgatacaagaaatactgggatggcgacggatttaattaatacatctcaagcatacgaccctaattcatcttttactgagttagctagtaaggtaatttgggtatttcctaatgctacaaagataacaactaaaggttatggtgtagtaggtagtgtgtttgcacctaatgcagttgtagaaacaaaaggtggttctattaatggacaagcctatgttggaggattacaccaaagagacggttttgaggttcataactttaaatttaactggccaaagtggaaaaagccagcagctaaaaaaggaaatttacaaattaaaaaagttgatgaaaacaatgaaaatattgttttaaaagacgcaaaatttgatgttatagataaagagaataatgttgtagatacggttacaacaaacgaaaagggtattgcggaagttaaagatttaccatttggtgattattttgtaaaagaaattagtgcaccagaaggatatataaagattgatgcaccagtaaaagtaacaattgataatacaaacataattgaatttgttataaagaatacgaaaaaattagaaaatggtcaattaaaattattgaaaaaagatagtgaatctggtcaacttctaccaggtgcaaaatttgatgttatcgataaagatgggaaagttgtggaaacaattgttacagatgataaaggtgaagctttatcgaaacaacttccagttggaagctatacattaaaagaagtagaagcaccgaaaggatatgaattatcatctagttcagtttctgttgatgtagaggctaataaagtagtgactgtagatgtggtgaataaaaagatccccgaaaaagtaacaggtcaatttgaagtggtgaaagtagatgcaaatgataaaacgaaattgttatcaggtgcagaattcgaagtgtataaagatggcaaaaaggtagcagaactgaaaacaggtgagagtggaaaagtgatgtcaccgaaattaccgctaggtgaatacacagtgaaagaaacgaaagcaccagcgggctacaagctttcagataaagaatggaaagtaacaattcaaaacgagaaagaagtagtaaaagtagaggcagaaaacgaaaaaatcttaggttctctacaaattattaaaatggatgataaagatcaaacgaaacgcttagcaggcgcagaatttacattgaaagatgtgaaaggcaatgttgtaaaagaaggaattacaacagataagtctggaactgttaaagtagacggacttgtgccgggtgaatatacgttagaagaaacaaaagcgccagaaggttataaggcattagaagtaacaatcgaagtaaacgtagtagcaaacgaagtagtaaaacaagacgtgttgaatgaaaaagtgaaagaagaaattacagggcaattagaaattacaaaggtagatgctaatgatataaataaaaaattagcaggcgcagtgtttgaaatttggaaagacggaacaaaaatcgatacattaacatcagatgaaaatggtaaagcaatttcgaaagaa

SEQ ID NO: 16 - DNA, 1002 bp, Bacillus anthracis
  nucleotide sequence of collagen-binding region A of BA5258
aacagggaaacatataagatggattggagctatagtaattcgaaacagcgtgaaataaagaccgaaataattaaaacggcttcggggagtattgcgtattgtttaacgcctgatttacgttctcccaatggagaagatttgcctgaaatggggaaaacatctgatgccgtatatcgtgttttattaaatggatatcctcaaaaaggcccatctgaattaggtgtagcgacaacggaagaagcgcattatgctacacaattagcagtatggattgcagcaaatgaattaacggaagaagatttagttgcaaaaaatgaacgtgtacataatcttatgaagcgtttagtagaggcttcaaagaaagaaactggatcacaagatgtattctttaaagttaatcctgtagattcacaaactgctacacaaaatggtgattatttagaaacaggattttatgcagtgcaaacaaatgcggtttctggctcttatacaattcttcctgaaaatgctcctaaagggcttcgaattgtaaatgaaaatggtgaagagaaaagtacgttatcaattaacgaaaaattcaaaattttacttccgaaagatacgagtagtggtaactttaaaatgaaagtgaagtctactttaacaaatttacaagcaatagcttttaaaggatcagaaaaagttcaaaatacaacggtattgttacaaagaaatagtgagaaaatcagtacagatttagttgtaaattgggaatcagtaggctctttaaagattatgaaattaggagaaaaaaaggaagtattaaaaggagcggtgtttgaagtttcgaatgaaaactttaaacaaaatgttacgactagtgataaggggattgcggagttaggtaatcttccaatcggtatatatagcgtcaaagaaattcaagcgccagctggatatgtgttagacagaagtgttaaaaaaattgaagtgaaaactggtgaaactgctgtattagagctg

SEQ ID NO: 17 - protein, 736 aa, Artificial sequence
  amino acid sequence of the His-tag fusion protein rBA0871A
MRGSHHHHHHGSTELKGLGDVSYYNAIIFGDHSATSADIEGAMAIQKNMNASSYTVLAAATGANNLAGATWVDEGYPSLLLGGQFTKAGAGQVIIQDGTVAMTKDGDPDSAMKTSYDRISYKEQAEIDAKFKEFRKDVDGVIGDASKLQTDKPKPNMSFGIGEDVNNPNIYVSSGQTGKKAFDVTDVFLPNVENKDFIVIYSDAEEVSFGSGAILYDTRNTGMATDLINTSQAYDPNSSFTELASKVIWVFPNATKITTKGYGVVGSVFAPNAVVETKGGSINGQAYVGGLHQRDGFEVHNFKFNWPKWKKPAAKKGNLQIKKVDENNENIVLKDAKFDVIDKENNVVDTVTTNEKGIAEVKDLPFGDYFVKEISAPEGYIKIDAPVKVTIDNTNIIEFVIKNTKKLENGQLKLLKKDSESGQLLPGAKFDVIDKDGKVVETIVTDDKGEALSKQLPVGSYTLKEVEAPKGYELSSSSVSVDVEANKVVTVDVVNKKIPEKVTGQFEVVKVDANDKTKLLSGAEFEVYKDGKKVAELKTGESGKVMSPKLPLGEYTVKETKAPAGYKLSDKEWKVTIQNEKEVVKVEAENEKILGSLQIIKMDDKDQTKRLAGAEFTLKDVKGNVVKEGITTDKSGTVKVDGLVPGEYTLEETKAPEGYKALEVTIEVNVVANEVVKQDVLNEKVKEEITGQLEITKVDANDINKKLAGAVFEIWKDGTKIDTLTSDENGKAISKE

SEQ ID NO: 18 - protein, 347 aa, Artificial sequence
  amino acid sequence of the His-tag fusion protein rBA5258A
MRGSHHHHHHGSMNRETYKMDWSYSNSKQREIKTEIIKTASGSIAYCLTPDLRSPNGEDLPEMGKTSDAVYRVLLNGYPQKGPSELGVATTEEAHYATQLAVWIAANELTEEDLVAKNERVHNLMKRLVEASKKETGSQDVFFKVNPVDSQTATQNGDYLETGFYAVQTNAVSGSYTILPENAPKGLRIVNENGEEKSTLSINEKFKILLPKDTSSGNFKMKVKSTLTNLQAIAFKGSEKVQNTTVLLQRNSEKISTDLVVNWESVGSLKIMKLGEKKEVLKGAVFEVSNENFKQNVTTSDKGIAELGNLPIGIYSVKEIQAPAGYVLDRSVKKIEVKTGETAVLEL

(19) United States
(12) Patent Application Publication
(10) Pub. No.: US 2006/0198852 A1
(43) Pub. Date: Sep. 7, 2006

(54) Collagen-binding MSCRAMMs of Bacillus anthracis and uses therefor

(76) Inventors: Magnus Hook, Houston, TX (US); Yi Xu, Houston, TX (US)

Correspondence Address: Benjamin Aaron Adler;ADLER & ASSOCIATES, 8011 Candle Lane, Houston, TX, 77071, US

(21) Appl. No.: 11/158,163

(22) Filed: Jun. 21, 2005

Related U.S. Application Data
(60) Provisional application No. 60/581,381, filed on Jun. 22, 2004.

(51) Int. Cl. C07K 14/32; C12N 1/21; C07H 21/04; C12N 15/74; A61K 39/02

(52) U.S. Cl. 424/190.1; 435/69.3; 435/252.31; 435/471; 530/350; 536/23.7

(57) ABSTRACT

The present invention provides cell wall anchored proteins of Bacillus anthracis representative of the MSCRAMM family of proteins and DNAs encoding the same. Also provided are collagen-binding peptides comprising the collagen-binding region A of the cell wall anchored proteins and DNAs encoding these peptides. Further provided are pharmaceutical compositions and immunogenic compositions thereof the cell wall anchored proteins, collagen-binding peptides and encoding DNAs. The immunogenic compositions are useful in methods of inducing an immune response against Bacillus anthracis.

### CROSS-REFERENCE TO RELATED APPLICATIONS

This is non-provisional claims benefit of provisional application U.S. Ser. No. 60/581,381, filed Jun. 22, 2004, now abandoned.

### FEDERAL FUNDING LEGEND

This invention was produced in part using finds obtained through Grants U54 AIO20624-21 and AR44415 from the National Institutes of Health. Consequently, the federal government has certain rights in this invention.

### BACKGROUND OF THE INVENTION

1. Field of the Invention

The present invention relates generally to the fields of protein molecular biology and pathogenic microbiology. More specifically, the present invention provides immunogenic compositions comprising MSCRAMM proteins, peptides or DNAs encoding the same of Bacillus anthracis and methods of use.

2. Description of the Related Art

The molecular pathogenesis of bacterial infections consists of multi-stage processes involving many different factors. Bacillus anthracis, a spore-forming Gram-positive organism that causes anthrax, is not an exception. Anthrax is initiated by the entry of spores into the host through inhalation, ingestion or via cuts in the skin. The spores are engulfed by macrophages, germinate, become vegetative bacilli that are capable of producing toxins, and disseminate in the host. They eventually reach the blood circulation where they multiply to a density of 10⁷-10⁸ cfu/ml, causing massive septicemia and toxemia.

In order to successfully establish an infection, B. anthracis must survive attacks from the host defense system and successfully colonize different tissues. The known principal virulence factors of B. anthracis are the two exotoxins, lethal toxin and edema toxin, and the poly-D-glutamic acid capsule. The toxins are thought to be largely responsible for the morbidity and mortality associated with anthrax while the capsule has been thought to have antiphagocytic activity and be necessary for in vivo survival (1-3). However, the processes by which germinated bacilli colonize different tissues and cross various barriers in the host to reach the bloodstream while avoiding being killed in the process remain unknown. Furthermore, the three forms of anthrax, i.e., cutaneous, gastrointestinal and inhalational, are likely to involve different sets of virulence factors.

Collagens are major components in the connective tissue and the most abundant proteins in mammals. There are over 20 types of collagen, among which type I collagen is a major component of the skin. It is not surprising that many bacteria have evolved to produce collagen adhesins to interact with this group of proteins, e.g., CNA of Staphylococcus aureus (4,5), YadA of Yersinia enterocolitica (6), FimH of meningitis-associated Escherichia coli O18acK1H7 (7), ACE of Enterococcus faecalis (8), Acm of Enterococcus faecium (9), CNE of Streptococcus equi (10), and RspA/RspB of Erysipelothrix rhusiopathia (11). As have been demonstrated for CNA (4,5,12-17) and YadA (18,19) in various animal models, these interactions can be critical in the establishment and progression of bacterial infections. It has been demonstrated that mice infected with S. aureus strains expressing CNA initially had similar numbers of S. aureus in the joints as mice infected with an isogenic S. aureus strain that expressed a mutated inactive form of CNA; however, as the infection progressed, the former group of mice showed significantly more S. aureus in the joints than the latter group as early as 24 hours post-inoculation (17).

Thus it seems that the adhesins allow the bacteria to “hold on” to tissue structures containing their corresponding ligand and as a result, these adhering bacteria appear to resist clearance by the host defense system. In addition, recombinant fragments of CNA and the recently reported RspA protected mice against challenge by wild type S. aureus (20) and E. rhusiopathia (11), respectively, raising the possibility that these proteins can be used as vaccine targets and underlining their importance in bacterial pathogenesis. Sequence analyses have also identified CNA-like proteins in other bacteria such as Bacillus spp., and Clostridium spp.; however, no functional studies of these proteins have been reported.

Among the collagen adhesins of Gram-positive organisms, CNA of S. aureus is the best characterized. CNA is a cell wall anchored protein (CWAP) that belongs to the MSCRAMM (Microbial Surface Component Recognizing Adhesive Matrix Molecules) family of adhesins. It has a domain organization typical of MSCRAMMs from Gram-positive bacteria; a signal peptide sequence at the N-terminus, a non-repetitive A region followed by one to four B repeats depending on the strains and a cell wall anchoring region, including an LPXTG-motif, a transmembrane segment and a short cytoplasmic tail rich in positively charged residues. The LPXTG motif is recognized by sortase A, a transpeptidase that cleaves the bond between T and G, and covalently links the T residue to the peptidoglycan in the cell wall. The A region is responsible for the collagen binding ability of CNA, while the B repeats are thought to help display the binding domain on the surface of staphylococci (4).

Structural analysis as well as comparison with other MSCRAMMs suggested that the A region of CNA consists of three subdomains rich in □-sheets and fold into immunoglobulin-like (Ig-like) domains. The middle subdomain in the CNA A region provides a trench-like hydrophobic surface in one of the □-sheets that can accommodate a triple helical collagen structure as indicated by molecular modeling experiments (21). Mutations of some of the residues in the postulated collagen-binding trench on CNA abolished or greatly reduced the collagen binding ability of the MSCRAMM. However, these residues are not necessarily conserved in the collagen binding A region of ACE (8), or the recently described RspA and RspB (11), suggesting differences in the detailed binding mechanisms of these molecules. The Ig-like folded subdomains have also been found in the binding A regions of other MSCRAMMs, such as the fibrinogen binding protein ClfA of S. aureus (22) and SdrG of S. epidermidis (23). Interactions between the subdomains are believed to be an integral part of the binding mechanisms of these molecules (23).

Although B. anthracis has been studied for over a hundred years, efforts have been mainly focused on elucidating the molecular mechanisms of the toxins and the capsule (1-3,36-38), which likely come into play in the later stages of the infection (39). Little is known regarding the early events in the establishment of anthrax. In addition, different factors are likely to be involved in the early stages of the three forms of anthrax. Adhesins that potentially could participate in the pathogenic process have not previously been identified in B. anthracis.

There is a need in the art for functional and structural characterization of MSCRAMMs of Bacillus anthracis and for elucidating the infection mechanisms of B. anthracis. Specifically, the prior art is deficient in the lack of cell wall anchored proteins or adhesin proteins of Bacillus anthracis effective to prevent infection thereby. The present invention fulfills this long-standing need and desire in the art.

### SUMMARY OF THE INVENTION

The present invention is directed to isolated DNA encoding a cell wall anchored protein of B. anthracis. The DNA comprises (a) isolated DNA which encodes a cell wall anchored protein of Bacillus anthracis; (b) isolated DNA which hybridizes under high stringency conditions to the isolated DNA of (a) above and which encodes a cell wall anchored protein of Bacillus anthracis; or (c) isolated DNA differing from the isolated DNAs of (a) or (b) above in codon sequence due to the degeneracy of the genetic code and which encodes a cell wall anchored protein. The present invention is directed to a related isolated DNA having a sequence shown in SEQ ID NO: 10 or SEQ ID NO: 11.

The present invention also is directed to an isolated and purified cell wall anchored protein of B. anthracis encoded by a cell wall anchored protein encoding DNA described herein. The present invention is directed further to a related isolated and purified protein having the sequence shown in SEQ ID NO: 12 or SEQ ID NO: 13.

The present invention is directed further to an isolated DNA encoding a collagen-binding region of a cell wall anchored protein of B. anthracis. The DNA comprises (a) isolated DNA which encodes a collagen-binding region of a cell wall anchored protein of Bacillus anthracis; (b) isolated DNA which hybridizes under high stringency conditions to the isolated DNA of (a) above and which encodes a collagen-binding region of a cell wall anchored protein of Bacillus anthracis; or (c) isolated DNA differing from the isolated DNAs of (a) or (b) above in codon sequence due to the degeneracy of the genetic code and which encodes a collagen-binding region of a cell wall anchored protein. The present invention is directed to a related isolated DNA having a sequence shown in SEQ ID NO: 15 or SEQ ID NO: 16.

The present invention is directed further yet to an isolated and purified collagen-binding peptide encoded by a collagen-binding region encoding DNA described herein. The present invention is directed to a related collagen-binding peptide having a sequence shown in SEQ ID NO: 15 or SEQ ID NO: 16.

The present invention is directed further still to pharmaceutical compositions and immunogenic compositions comprising the DNAs, cell-wall anchored proteins and collagen-binding peptides described herein.

The present invention is directed further still to a method of inducing a host-mediated immune response against Bacillus anthracis in a subject. The method comprises administering the immunogenic composition described herein to a subject whereby host immune cells are activated against the cell wall anchored protein or collagen-binding protein described herein or encoded by a DNA described herein comprising the immunogenic composition. Subsequent presentation of the cell wall anchored protein or the collagen-binding peptide by Bacillus anthacis in the subject induces the host-mediated immune response against Bacillus anthracis.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1A depicts the gene organization of the regions encoding BA0871 and BA5258. The direction of transcription of each gene is indicated by the direction of the arrow at the end of each block. Lollipops indicate putative transcription terminators. FIG. 1B depicts the domain organization of BA0871 and BA5258. BA0871 and BA5258 proteins share similar domain organization as CNA, i.e., a signal peptide at the N-terminus (black-shaded boxes), a non-repetitive A region (white boxes), repeats (hatched boxes) and a cell wall anchoring region containing LPXTG (SEQ ID NO: 1) motif at the C-terminus. Regions containing Ig-like folds are indicated by arrows above the proteins. Numbers below the boxes and above the arrows indicated the position of amino acid residues. Dotted lines define the boundary of the homologous region between CNA and BA0871, and the dashed lines the homologous region between CNA and BA5258.

FIG. 2A is an SDS-PAGE of purified recombinant A regions BA0871 (rBA0871A) and BA5258 (rBA5258A) FIG. 2B depicts the far-UV circular dichroism spectra of rBA0871A and rBA5258A. Proteins were in 1% PBS, pH 7.4. Spectra were recorded at room temperature in a 0.05-cm cuvette. The spectrum of 1% PBS was subtracted.

FIGS. 3A-3B demonstrate dose-dependent binding of rBA0871A and rBA5258A. Increasing concentrations of digoxigenin labeled rBA0871A (FIG. 3A) and rBA5258A (FIG. 3B) were incubated with immobilized bovine type I collagen or BSA. Bound proteins were detected with anti-digoxigenin-AP Fab fragment (1:5000 dilution).

FIGS. 4A-4F depicts the surface plasmon resonance analysis of the binding of rBA0871A and rBA5258A to bovine type I collagen. Recombinant proteins were flown over a surface coated with collagen. Representative sensorgrams of increasing concentrations of rBA0871A (FIG. 4A) and rBA5258A (FIG. 4B) are shown. The dissociation constants were determined by Scatchard plot analysis (FIGS. 4C and 4D for rBA0871A and rBA5258A, respectively) and one-site-binding nonlinear regression analysis (FIGS. 4E and 4F for rBA0871A and rBA5258A, respectively). □_{bound}, the binding ratio; and [P]_{free}, the concentration of free protein.

FIGS. 5A-5B demonstrate expression of the A regions of BA0871 and BA5258 on the surface of S. carnosus strain TM300. Exponential phase cells were incubated with lysostaphin in the presence of protease inhibitors. The cells were then centrifuged and the supernatants were subjected to western blot analysis. Supernatants were probed with mouse anti-BA0871 sera (1:1000 dilution) (FIG. 5A) or with mouse anti-BA5258 sera (1:1000 dilution) (FIG. 5B). Goat-anti-mouse IgG-AP conjugant was used as the secondary antibody. Arrows indicate bands of the expected sizes.

FIG. 6 demonstrates the attachment of S. carnosus expressing BA0871 and BA5258 to a collagen-coated surface. Exponential phase cells were incubated with immobilized bovine type I collagen or BSA. Bound cells were fixed with formaldehyde and stained with crystal violet. Absorbence at 590 nm was measured. Filled squares, TM300(BA0871A) with collagen; filled inverted triangles, TM300(BA5258A) with collagen; filled circles, TM300(pYX105) with collagen; open squares, TM300(BA0871A) with BSA; open inverted triangles, TM300(BA5258A) with BSA; and open circles, TM300(pYX105) with BSA.

### DETAILED DESCRIPTION OF THE INVENTION

In one embodiment of the present invention there is provided an isolated DNA encoding a cell wall anchored protein of B. anthracis comprising (a) isolated DNA which encodes a cell wall anchored protein of Bacillus anthracis; (b) isolated DNA which hybridizes under high stringency conditions to the isolated DNA of (a) above and which encodes a cell wall anchored protein of Bacillus anthracis; or (c) isolated DNA differing from the isolated DNAs of (a) or (b) above in codon sequence due to the degeneracy of the genetic code and which encodes a cell wall anchored protein.

In one aspect of this embodiment the DNA may have the nucleotide sequence shown in SEQ ID NO: 10 or SEQ ID NO: 11. In this aspect the cell wall anchored protein may have the amino acid sequence shown in SEQ ID NO: 12 or SEQ ID NO: 13. Further to this aspect the cell wall anchored protein may have a collagen-binding region having a sequence shown in SEQ ID NO: 17 or SEQ ID NO: 18. In another aspect of this embodiment the cell wall anchored protein encoding DNA may have a sequence that is about 90% homologous to the nucleotide sequence shown in SEQ ID NO: 10 or SEQ ID NO: 11. In a related embodiment the present invention provides a vector comprising the isolated cell wall anchored protein encoding DNA described supra and the regulatory elements necessary for expression of the DNA on the surface of a bacterium. Further to this related embodiment there is provided a host bacterium comprising and expressing this vector.

In another related embodiment the present invention provides a pharmaceutical composition comprising the cell wall anchored protein encoding DNA described supra and a pharmaceutically acceptable carrier. In yet another related embodiment there is provided an immunogenic composition comprising an immunogenically effective amount of the cell wall anchored protein encoding DNA and a pharmaceutically acceptable carrier, adjuvant or diluent or a combination thereof.

In still another related embodiment there is provided a method of inducing a host-mediated immune response against Bacillus anthracis in a subject, comprising administering the immunogenic composition comprising the cell wall anchored protein encoding DNA described supra to the subject, wherein the cell wall anchored protein expressed by the DNA comprising the immunogenic composition is effective to activate host immune cells against the protein such that subsequent presentation of the protein by Bacillus anthracis in the subject induces the host-mediated immune response against Bacillus anthracis. In an aspect of this embodiment the DNA may comprise a vector effective to express the DNA.

In another embodiment of the present invention there is provided an isolated and purified cell wall anchored protein of B. anthracis encoded by the cell wall anchored protein encoding DNA described supra. In an aspect of this embodiment the isolated and purified cell wall anchored protein of may have the sequence shown in SEQ ID NO: 12 or SEQ ID NO: 13. Further to this aspect the cell wall anchor protein may comprise a collagen-binding region having a sequence shown in SEQ ID NO: 15 or SEQ ID NO: 16. In another aspect the cell wall anchored protein may have a sequence that is about 90% homologous to the nucleotide sequence shown in SEQ ID NO: 12 or SEQ ID NO: 13.

In a related embodiment the present invention provides a pharmaceutical composition comprising the cell wall anchored protein described supra and a pharmaceutically acceptable carrier. In yet another related embodiment there is provided an immunogenic composition comprising an immunogenically effective amount of the cell wall anchored protein and a pharmaceutically acceptable carrier, adjuvant or diluent or a combination thereof.

In still another related embodiment there is provided a method of inducing a host-mediated immune response against Bacillus anthracis in a subject, comprising administering the immunogenic composition comprising the cell wall anchored protein described supra to the subject, wherein the cell wall anchored protein comprising the immunogenic composition is effective to activate host immune cells against itself such that subsequent presentation of the protein by Bacillus anthracis in the subject induces the host-mediated immune response against Bacillus anthracis.

In yet another embodiment of the present invention there is provided a isolated DNA encoding a collagen-binding region of a cell wall anchored protein of B. anthracis comprising (a) isolated DNA which encodes a collagen-binding region of a cell wall anchored protein of Bacillus anthracis; (b) isolated DNA which hybridizes under high stringency conditions to the isolated DNA of (a) above and which encodes a collagen-binding region of a cell wall anchored protein of Bacillus anthracis; or (c) isolated DNA differing from the isolated DNAs of (a) or (b) above in codon sequence due to the degeneracy of the genetic code and which encodes a collagen-binding region of a cell wall anchored protein. In an aspect of this embodiment the DNA may have the nucleotide sequence shown in SEQ ID NO: 15 or SEQ ID NO: 16. In another aspect the collagen-binding region encoding DNA may have a sequence that is about 90% homologous to a sequence shown in SEQ ID NO: 15 or SEQ ID NO: 16.

In a related embodiment the present invention provides a vector comprising the isolated collagen-binding region encoding DNA described supra and the regulatory elements necessary for expression of the DNA on the surface of a bacterium. Further to this related embodiment there is provided a host bacterium comprising and expressing this vector. In other related embodiments there are provided pharmaceutical and immunogenic compositions each comprising the collagen-binding region encoding DNA and pharmaceutically acceptable carriers, adjuvant or diluent or a combination thereof as as described supra.

In still another related embodiment there is provided a method of inducing a host-mediated immune response against Bacillus anthracis in a subject using the immunogenic composition comprising the collagen-binding region encoding DNA as described supra. The collagen-binding region encoding DNA may comprise a vector as described supra.

In still another embodiment of the present invention there is provided an isolated and purified collagen-binding peptide encoded by the DNA encoding the collagen-binding region described supra. In one aspect of this embodiment the collagen-binding peptide may have the sequence shown in SEQ ID NO: 17 or SEQ ID NO: 18. In another aspect the collagen-binding peptide may have a sequence that is about 90% homologous to a sequence shown in SEQ ID NO: 17 or in SEQ ID NO: 18.

In related embodiments there are provided pharmaceutical and immunogenic compositions each comprising the collagen-binding peptide and pharmaceutically acceptable carriers, adjuvant or diluent or a combination thereof as described supra. In still another related embodiment there is provided a method of inducing a host-mediated immune response against Bacillus anthracis in a subject using the immunogenic composition comprising the collagen-binding region encoding DNA as described supra.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof. As used herein, the term “subject” may mean an individual or more than one individual as comprises a population of individuals. The individual may be a human or non-human animal.

Provided herein are cell wall anchored proteins (CWAPs) or adhesins of B. anthracis that structurally and functionally are similar to the family of CNA-like collagen binding MSCRAMMs. These B. anthracis cell wall anchored proteins have the domain organization typical of the MSCRAMM family of proteins, i.e., signal peptide sequences, a non-repetitive A region followed by repeats and a characteristic cell wall anchoring region. The A region is structurally and functionally effective to bind or otherwise interact with host matrix proteins, e.g., the triple helical structure of a collagen, and to mediate attachment to collagen when expressed on the surface of a heterologous host bacterium.

The present invention also provides DNAs encoding these cell wall anchored proteins or region A peptide fragments thereof of Bacillus anthracis. The DNA may be an isolated DNA which encodes the cell wall anchored proteins or region A peptides. These DNAs may have nucleotide sequences shown in SEQ ID NOS: 10 or 11 for the cell wall anchored proteins or nucleotide sequences SEQ ID NOS: 15 or 16 for the region A peptides. Alternatively, the DNA may be another isolated DNA that may have a nucleotide sequence that is substantially homologous to the isolated DNA encoding the cell wall anchored proteins or region A peptides. Preferably the sequence has a 70% homology to the isolated DNAs, more preferably an 80% sequence homology or most preferably a 90% sequence homology.

It is well known in the art that sequences are substantially homologous when at least about 70% or 75%, preferably at least about 80% and most preferably at least about 90% or 95% of the nucleotides match over the defined length of the DNA sequences. Sequence homology can be identified by comparing the sequences using standard software available in sequence data banks or, alternatively, in a Southern hybridization experiment under, for example, stringent conditions as defined for that particular system. Defining appropriate hybridization conditions and stringency is well within the skill of the art. Thus, also provided are vectors comprising any one of the CWAP or region A peptide DNAs and regulatory elements necessary for expression of the DNA in a cell. The invention is also directed toward host cells transfected with any of these a vector(s). The host cell may be any cell known and standard in the art that may be transfected with these vectors.

The B. anthracis cell wall anchored proteins may be encoded by any of the DNAs described herein. Particular proteins BA0871 and BA5258 have the amino acid sequences shown in SEQ ID NOS: 12 or 13, respectively. Alternatively, the cell wall anchored proteins may have sequences that are 70%, 80% or 90% homologous to SEQ ID NOS: 12 or 13. Particularly, BA0871 and BA5258 each comprise a collagen-binding A region. The respective sequences of these collagen-binding A regions are shown in SEQ ID NOS: 17 or 18. Therefore, the cell wall anchored proteins or region A peptides may have amino acid sequences that are homologous to the isolated cell wall anchored proteins or region A peptides. Preferably the amino acid sequence has a 70% homology to the isolated cell wall anchored proteins or region A peptide, more preferably an 80% sequence homology or most preferably a 90% sequence homology.

The present invention also provides pharmaceutical compositions comprising cell wall anchored proteins or collagen-binding peptides or DNA encoding the same. The pharmaceutical composition comprises any pharmaceutically acceptable carrier known and standard in the art. Formulations of the same are readily prepared by standard methods and well within the skill of the art.

It is contemplated that these cell wall anchored proteins or collagen-binding peptides or DNA encoding the same may be effective to induce a host-mediated response against Bacillus anthracis. These collagen-binding peptides may be used in the preparation of an immunogenic composition suitable to effect immunization of a subject against Bacillus anthracis. The immunogenic composition may comprise a carrier or a suitable adjuvant to boost immune response or a combination thereof, as are known in the art. The immunogenic composition further may comprise a diluent standard in the art. The immunogenic composition may comprise a vaccine. Thus, the effect of a vaccine comprising the immunogenic composition is vaccination or inoculation against B. anthracis whereby subsequent challenge with B. anthracis spores will elicit a host immune response against the organism to prevent or minimize infection.

The collagen-binding peptides may be produced recombinantly using standard molecular biological techniques or synthetically by standard protein synthetic methodologies. Alternatively, a genetic sequence encoding the collagen-binding peptides may be delivered as naked DNA to an individual via appropriate methods known in the art. Also, the genetic sequence may be introduced or inserted into a suitable vector, such as for example, but not limited to, attenuated viral or bacterial vectors, as are standard in the art. Thus, host cells, preferably a bacterium, comprising these vectors are also provided.

The immunogenic composition may be used to immunize, vaccinate or inoculate a subject or subject population at risk of infection by B. anthracis. Preferably, the subject is protected against cutaneous anthrax, although it is contemplated that a beneficial immunity against gastrointestinal and inhalational anthrax is acquired. As used herein, immunizing or immunization of a subject encompasses full and partial immunization whereby the subject becomes fully immune to the condition or partially immune to the condition. The subject may be a mammal, preferably a human.

Methods of administering the immunogenic compositions are well known and practiced by those of ordinary skill in the art. Furthermore, the effective dose needed to induce a host-mediated response in a subject or subject population is determined easily without undue experimentation. One of ordinary skill in the art could readily determine if administration of the proteins, peptides or DNAs encoding the same or immunogenic compositions is in a single dose or multiple doses. If necessary additional doses of the immunogenic compositions may be administered as a booster to the original immunizing or vaccinating dose.

The following examples are given for the purpose of illustrating various embodiments of the invention and are not meant to limit the present invention in any fashion.

### EXAMPLE 1

Bacterial Strains, Plasmids and Culture Conditions

Escherichia coli strains were grown at 37° C. overnight in Lennox L broth (LB) (Sigma) or on LB agar supplemented with ampicillin (50 g/ml) when appropriate. Staphylococcus carnosus strains were grown at 37C in tryptic soy broth (TSB) (Difco) or on tryptic soy agar (TSA) supplemented with chloramphenicol (Cm) (10 g/ml) when necessary. Bacillus anthracis Sterne strain 7702 was grown in LB at 37C.

### EXAMPLE 2

Identification of LPXTG-motif Containing Cell Wall Anchored Proteins

The genome of B. anthracis Ames strain was analyzed using a combination of a bioinformatics method as described previously (23,24), and searching annotated genome sequence (www.tigr.org) with terms “LPXTG” and/or “cell wall anchor”. Nine such proteins were identified.

### EXAMPLE 3

Cloning, Expression and Purification of Recombinant A Regions of BA0871 and BA5258

Genomic DNA of B. anthracis Sterne strain 7702 was prepared using the G NOME kit (BIO 101, Carlsbad, Calif.) according to manufacturer's instructions. DNA fragments encoding the A region (amino acid residues 42-765) of BA0871 and the A region (amino acid residues 32-366) of BA5258 were PCR-amplified from the genomic DNA preparation. Primer pair BA0871L, 5′ GAAGGATCCACAGAATTAAAAGGTTTAGGTG 3′ (SEQ ID NO: 2), and BA0871RPQE, 5′ GAAGTCGACTCATTCTTTCGAAATTGCTTTACC 3′ (SEQ ID NO:3), were used for BA0871. Primer pair BA5258L, 5′ GAAGGATCCAACAGGGAAACATATAAGATG 3′ (SEQ ID NO: 4), and BA5258RPQE, 5′ GAAGTCGACTCACAGCTCTAATACAGCAGTTTC 3′ (SEQ ID NO: 5), were used for BA5258. The underlined portion of the primer sequences indicates sites for restriction enzymes. The PCR products were cloned into pQE30 as described previously (16). Large-scale expression and purification of recombinant proteins were performed as described previously (25) using Ni²⁺—affinity chromatography and ion exchange chromatography. The sizes of the purified proteins were confirmed by mass spectrometry at Tufts Protein Chemistry Facility, Tufts University.

### EXAMPLE 4

Circular Dichroism (CD) Spectroscopy

Recombinant A regions of BA0871 and BA5258 were dialyzed against 1% phosphate buffered saline (PBS), pH 7.4. Their CD spectra were measured with a Jasco J720 spectropolarimeter at room temperature in a 0.05-cm cuvette as described previously (25). Data were integrated for 1 sec at 0.2-nm intervals with a bandwidth of 1 nm and 20 accumulations. Secondary structure compositions were estimated using five deconvulution programs, CD Estima (26), Contin (27), Neural Network (28), Selcon (29) and Varslcl (30). The results were averaged as described (31).

### EXAMPLE 5

Enzyme-Linked Immunosorbent Assays (ELISAs)

Proteins were labeled with digoxigenin (Roche) according to manufacturer's instructions and dialyzed against PBS, pH 7.4. ELISA based binding assays were performed based on the method described previously with slight modifications (25). Briefly, the wells of 96-well microtiter plates were coated with 1 g/well bovine type I collagen or bovine serum albumin (BSA) and then blocked with PBS containing 1% BSA and 0.1% Tween 20. Increasing concentrations of digoxigenin-labeled recombinant proteins were added to corresponding wells and incubated for 1-2 hours at room temperature. Bound proteins were detected with anti-digoxigenin-AP Fab fragment (Roche) (1:5000 dilution). Assays were performed in triplicates and results were reproducible. Data were presented as the mean value±S.E. of A_{405 nm} from a representative experiment. Apparent dissociation constants were determined using a one-site-binding nonlinear regression model (GraphPad Prism 4) as described previously (25).

### EXAMPLE 6

Surface Plasmon Resonance (SPR) Analysis of Collagen-Binding of Recombinant Proteins

SPR analysis was performed at ambient temperature in a BIAcore 3000 system (BIAcore AB, Uppsala, Sweden) based on the method described previously (25). Briefly, bovine type I collagen was immobilized onto the cells of a BIAcore CM5 sensor chip. For blank control, one of the cells on the chip was activated and deactivated in the same manner as for the immobilization of collagen except that buffer instead of collagen was used. Increasing concentrations of recombinant proteins in HEPES buffered saline (HBS) (10 mM HEPES, 150 mM NaCl, pH 7.4) were injected into the cells in the sensor chip at a flow rate of 30 l/min for 5 minutes. The surfaces were regenerated with 16 mM Tris, 1M NaCl, pH 8.5 or 15 mM Tris, 1 M NaCl, pH 9. Responses from the blank control were low and were subtracted from responses from the collagen surface. Analysis of the association and dissociation rates was performed using the BIAevaluation 3.0 software (BlAcore). Scatchard plot and nonlinear regression analysis (GraphPad Prism 4 software) was carried out using data from the equilibrium portion of the sensorgrams as described previously (25). Values for the binding ratio, □_{bound}, and the concentration of free proteins, [P]_{free}, were calculated based on the correlation between the SPR response and change in the mass of total bound proteins (25).

### EXAMPLE 7

Construction of a Staphyococcus carnosus Surface-Display Expression Vector

The E. coli—staphylococci shuttle vector pLI50 (32) was modified as follows: A fragment containing the promoter and signal peptide region of the cna gene of S. aureus was obtained by digesting plasmid pYX102 (17) with EcoRI and BglII. The fragment was cloned into pLI50 digested with EcoRI and BamHI to form pYX103. To introduce the cell wall anchoring motif into pYX103, the sequence encoding the last B repeat to 51 nucleotides 3′ of the end of cna gene was PCR-amplified using primer pairs CNAB5′, 5′GAAGTCGACACAACATCAATTAGTGGTG 3′ (SEQ ID NO: 6), and PSTCNA3′ 5′ AACCTGCAGTACATAGAACTAAGAATAGCC 3′ (SEQ ID NO: 7). The underlined portion of the primer sequences indicates sites for restriction enzymes. The product was cloned into pYX103, resulting in plasmid pYX105. The inserted regions were confirmed by DNA sequence analysis.

### EXAMPLE 8

Generation of S. carnosus Heterologous Strains

Primer pairs BA0871L and BA0871RYX105, 5′ GAAGTCGACTTCTTTCGAAATTGCTTTACC 3′ (SEQ ID NO: 8), and BA5258L and BA5258RYX105, 5′ GAAGTCGACCAGCTCTAATACAGCAGTTTC 3′, (SEQ ID NO: 9) were used to PCR-amplify regions encoding the A regions of BA0871 and BA5258, respectively. The underlined portion of the primer sequences indicates sites for restriction enzymes. The PCR products were cloned into pYX105. The ligation mixture was transformed into E. coli JM101. Transformants were verified by examining the DNA banding patterns using agarose gel electrophoresis of restriction digestions of plasmid preparations, and DNA sequence analysis.

Electrocompetent cells of S. carnosus strain TM300 were prepared by washing 200 ml of exponential phase TM300 cells with 200 ml ice-cold 0.5 M sucrose twice. The cells were then resuspended in 0.8 ml ice-cold 0.5 M sucrose, aliquoted and stored at −80° C. until ready to use. 50 l of thawed TM300 competent cells were mixed with 5 l plasmid DNA prepared from E. coli JM 101 clones with QIAprep Spin Miniprep Kit from a 5 ml overnight culture. Electroporation was performed in a BTX electroporation system with a 0.1 cm gap electroporation cuvetter (Bio-Rad) using the following parameters: 2.5 kV, 25 Fd, and 72Ω. Immediately after electroporation, 1 ml of tryptic soy broth was added to the cells. The mixture was incubated at 37C for 1 hour, plated onto TSB agar plates containing 10 g/ml Cm and incubated at 37C for 16-24 hours. Colonies were examined by use of PCR.

### EXAMPLE 9

Characterization of Heterologous S. carnosus TM300 Strains

Mouse anti-sera were obtained by intravenously injecting female Balb/c mice with recombinant proteins of BA0871 or BA5258. The mice were bled one and two weeks post injection. Lysostaphin was used to extract cell wall anchored proteins from heterologous TM300 strains as described (33). The extracts were subjected to western blot analysis probed with mouse anti-rBA0871 or mouse anti-rBA5258 sera to determine the surface expression of BA0871A and BA5258A as described (17).

Attachment of heterologous TM300 strains to bovine type I collagen was assayed as described previously (17). Various concentrations of log phase cells were incubated with immobilized bovine type I collagen (10 g/well). The wells were washed with PBS. Attached bacteria were fixed with 25% formaldehyde, and stained with 0.5% crystal violet. After washing, 100 μl of 10% acetic acid was added and the absorbance at 590 nm was measured. Assays were performed in triplicates and the results were reproducible. Data were presented as the mean value±S.E. of A_{590 nm} from a representative experiment.

### EXAMPLE 10

Identification and Sequence Analysis of BA0871 and BA5258 of B. anthracis

To identify putative CWAPs of B. anthracis, the genome of B. anthracis Ames strain was analyzed using a bioinformatics method developed in house (23,24). In addition, the annotated genome (www.tigr.org) was searched with terms “LPXTG” and/or “cell wall anchor”. The results of the two methods were combined and nine CWAPs were identified. Two of them, BA0871 and BA5258, have low-level sequence homology to CNA, the S. aureus collagen adhesin.

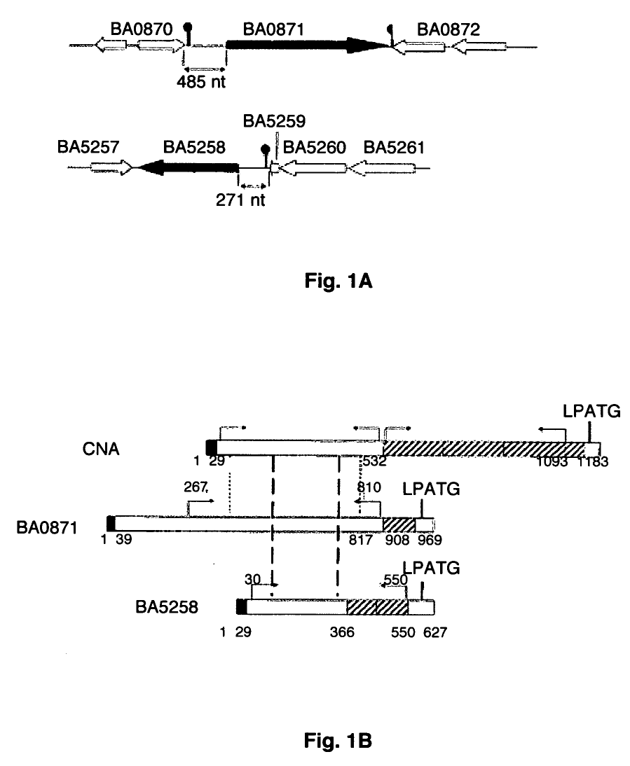

The BA0871 open reading frame (ORF) (SEQ ID NO: 10) is 2910 nucleotides long from position 877850 to 880759 on the chromosome of the Ames strain. Within the BA0817 ORF, a 2172 nt sequence beginning at nt 124 encodes the collagen-binding A region (SEQ ID NO: 15). The gene is flanked by BA0870, a putative hydrolase, 485 nt upstream at the 5′ end and by BA0872, a putative N-acetylmuramoyl-L-alanine amidase, 58 nt downstream at the 3′ end (FIG. 1A). BA0872 is transcribed from the opposite direction to BA0871. Putative transcription terminator sequences were identified 3′ of BA0870 and BA0871 ORFs, respectively. Therefore, the BA0871 gene does not appear to be in an operon. The BA5258 ORF (SEQ ID NO: 11) is 1884 nt long from position 4765673 to 4763790 on the chromosome of Ames. Within the BA5258 ORF a 1002 nt sequence beginning at nt 97 encodes the collagen-binding A region (SEQ ID NO:16). It is flanked by hypothetical genes at either end. A putative transcription terminator sequence was found between BA5257 (271 nt upstream of BA5258) and BA5258. At the 3′ side 31 nt downstream, ORF BA5259 is transcribed in the opposite direction to BA5258. Thus, BA5258 is also unlikely to be in an operon.

The deduced BA0871 protein (SEQ ID NO: 12) is 969 amino acids long with a calculated isoelectric point (pI) of 4.56. The predicted BA5258 protein (SEQ ID NO: 13) is 627 amino acids long with a calculated pI of 9.10. Examination of the amino acid sequences of the two proteins revealed that they have similar domain organization to that of CNA (FIG. 1B). Both contain signal peptide sequence at the N-terminus, a non-repetitive A region followed by repeats. At the C-terminus, they contain typical cell wall anchoring sequences: an LPXTG-motif, i.e., LPATG (SEQ ID NO: 14), a transmembrane segment and a short cytoplasmic region with positively charged residues.

After post-translational processing by signal peptidase and sortase, the mature proteins of BA0871 and BA5258 have calculated molecular weights of 102713.2 (res. 40-941) and 62804.1 (res. 30-594), respectively. The repeat region in BA0871 (res. 818-908) consists of 13 tandem repeats of a 7 amino acid residues long unit, while the repeat region (res. 367-551) in BA5258 consists of two tandem repeats of 94 a.a. and 91 a.a., respectively. Secondary structure prediction using PHDsec at the PredictProtein server (http://cubic.bioc.columbia.edu/predictprotein/) indicated that the A regions of both proteins are mainly □-sheets and loops. Fold predication using 3D-PSSM web server (http://www.sbg.bio.ic.ac.uk/˜3dpssm/) showed that residues 267-810 in the A region of BA0871 and the entire mature protein of BA5258 are highly likely to adopt Ig-like folds, with probability E values of 7.33e-06 and 0.0176, respectively. The similarities between CNA and the two proteins are not high, however, the homologous regions cover relatively long areas in the proteins; a 357 amino acid-long stretch with 24% identity and 39% similarity between the A regions of CNA and BA0871 and a 183 amino acid-long stretch with 26% identity and 41% similarity between the A regions of CNA and BA5258 (FIG. 1B). Thus, BA0871 and BA5258 appear to belong to the family of CNA-like MSCRAMMs.

### EXAMPLE 11

Expression Purification and Secondary Structure Analysis of the A Regions of BA0871 and BA5258

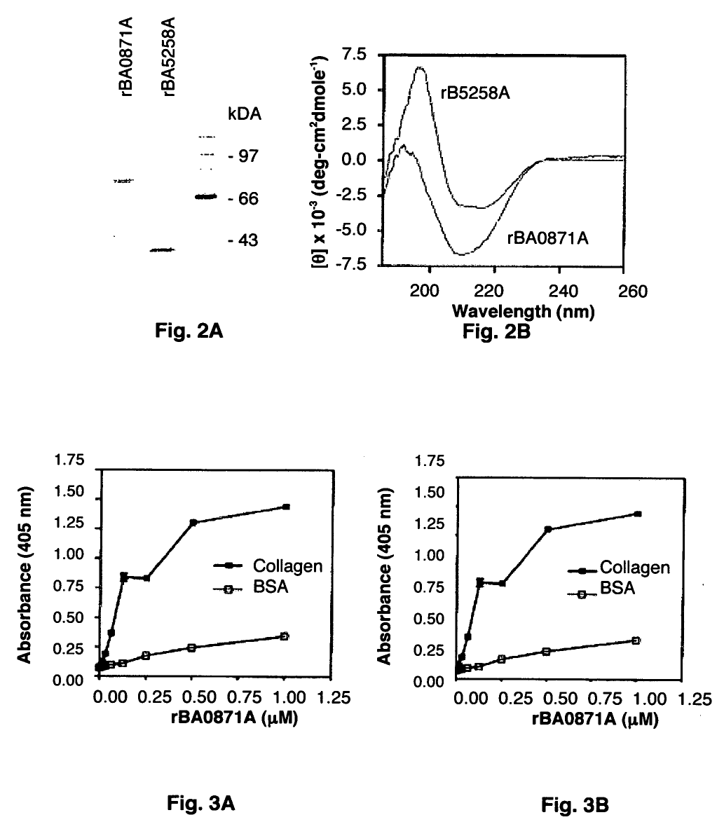

The predicted A regions of each protein were expressed in E. coli as His-tag fusion proteins rBA0871A (SEQ ID NO: 17) and rBA5258A (SEQ ID NO: 18). Attempts to purify the two recombinant fusion proteins from E. coli lysates by metal chelating chromatography were unsuccessful because both proteins had weak affinity for the nickel column and were present in the flow through, wash buffer and early fractions of the eluant. The latter two groups were pooled and the recombinant proteins were further purified using ion-exchange chromatography, which yielded proteins of reasonably high purity. The calculated molecular weights for rBA0871A and rBA5258A are 80029.3 Da and 38383.4 Da, respectively. The two proteins migrated at approximately the expected sizes on SDS-PAGE (FIG. 2A). Mass spectrometry analysis of the two purified proteins indicated that their masses were 80030.9 and 38382.2, respectively, in good agreement with the calculated molecular weights. A smaller, weaker band could be observed in the purified rBA5258A sample; however, it was not detected by mass spectrometry and could be due to a subpopulation of the full-length recombinant protein being more compactly folded.

To determine the secondary structure composition of the A regions of BA0871 and BA5258, the recombinant proteins were analyzed by CD spectroscopy (FIG. 2B). Deconvolution of the spectra indicated that both proteins are predominantly [□sheets. Recombinant BA0871A has 8.3±2.8% □-helices, 51.0±6.1% □-sheets and 30.8±18.9% other random structures and rBA5258A has 8.0±1.0% □-helices, 63.3±6.9% □-sheets and 28.7±17.0% other random structures. These compositions are similar to the A regions of the MSCRAMMs of staphylococci and enterococci (8,21-24,34,35).

### EXAMPLE 12

Analysis of the Binding of the A Regions of BA0871 and BA5258 to Type I Collagen

To determine the binding capabilities of the two proteins, solid phase binding assays were performed. Both rBA0871A and rBA5258A bound bovine type I collagen, but not BSA, in a dose-dependent and saturable manner (FIGS. 3A-3B). The apparent dissociation constants (K_{Dapp}) are 0.19±0.04 M for rBA0871A and 0.03±0.003 M for rBA5258A, respectively.

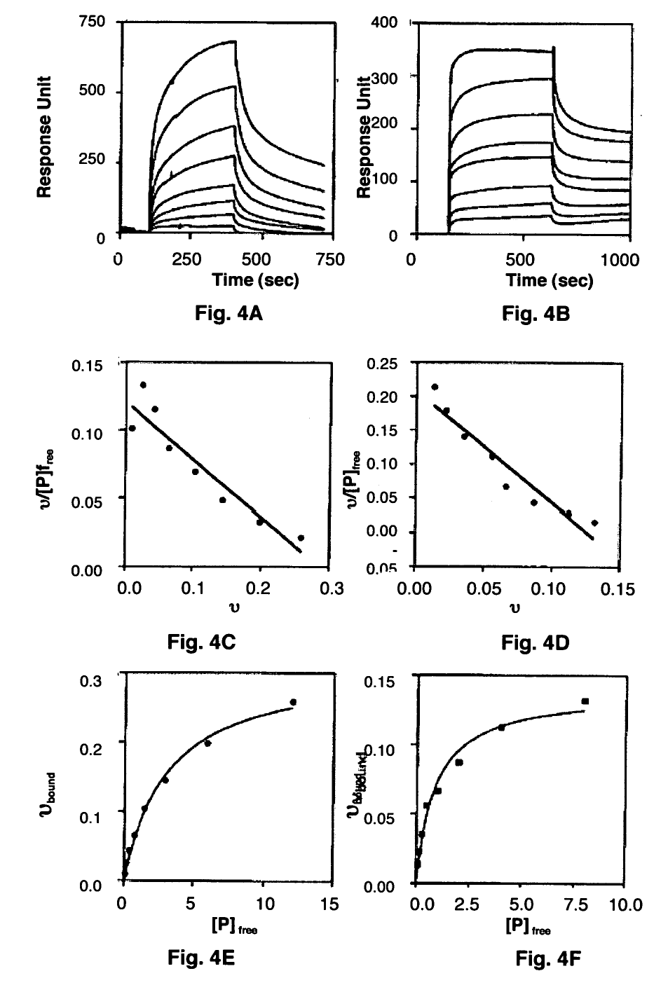

The binding of the recombinant B. anthracis proteins to collagen was further analyzed by surface plasmon resonance (SPR) using a BIAcore 3000 system. Examination of the sensorgrams for the two proteins indicated that they exhibited different kinetics for binding to immobilized type I collagen with rBA0871A showing markedly slower association and dissociation rates than rBA5258A (FIGS. 4A-4B). The association rate, kₐ, and dissociation rate, k_{d}, for rBA0871A were calculated to be 2.8±1.4×10³ M⁻¹s⁻¹ and 4.3±1.9×10⁻³ s⁻¹, respectively, resulting in a dissociation constant, K_{D}, of 1.6±0.1 M. Scatchard analysis based on the responses at the equilibrium portion of the rBA0871A sensorgrams (25) indicated a mostly linear distribution of the data points with a K_{D} of around 2.4 M (FIG. 4C). The K_{D} of rBA0871A for type I collagen was also calculated using a one-site-binding nonlinear regression model and a K_{D} of 3.2±0.4 M was obtained (25) (FIG. 4E). Thus, the dissociation constant for the interaction of rBA0871A with type I collagen is ˜1.6−3.2 M.

In contrast, rBA5258A associates and dissociates with type I collagen much faster, the kₐ and k_{d} for rBA5258A binding to type I collagen are in fact too rapid to be determined accurately and, therefore, are not reported. To calculate the K_{D} of rBA5258A for type I collagen, the responses at the equilibrium portion of the sensorgrams were first analyzed by Scatchard plot (FIG. 4D). The data points formed a slightly concaved shape; however, fitting the low and the high concentration ranges separately did not give sufficiently different K_{D} values and therefore the entire concentration range was fitted linearly, giving a K_{D} of around 0.6 M. The binding affinity was also calculated to be 0.9±0.2 M using a one-site-binding nonlinear regression model (FIG. 4F). Thus, the K_{D} determined for the interaction of rBA5258A with type I collagen using SPR is ˜0.6−0.9 M.

This binding analysis indicates that rBA0871A and rBA5258A specifically bind type I collagen in a dose-dependent manner with rBA5258A exhibiting higher affinity for collagen than rBA0871A in both SPR analyses and solid phase binding assays. The K_{Dapp} values for the interactions between type I collagen and the two recombinant proteins obtained from sold phase binding assays are lower than the K_{D} values obtained from SPR analyses. This could be due to the intrinsic differences between the two methods and has been observed in the binding analyses of other MSCRAMMs.

Both rBA0871A and rBA5258A showed a relatively simple binding model with one affinity binding class, similar to that observed for the collagen binding A domain of ACE, the E. faecalis collagen adhesin (K_{D}=˜48 _M) (8), but unlike the CNA A domain which exhibited a range of binding affinities for type I collagen (K_{D} values ˜0.21−35 μM) (40). The differences in the dissociation constants and binding affinity classes suggest that the specific molecular interactions between type I collagen and the four collagen-binding MSCRAMMs are somewhat different. Kinetics analysis shows that the two B. anthracis proteins have very different association and dissociation rates. The binding kinetics of rBA0871A resembles that of CNA with relatively slow association and dissociation rates (40), whereas rBA5258A resembles that of ACE with rapid association and dissociation (8). This also suggests that the detailed collagen binding mechanisms of the two B. anthracis proteins are different. It is contemplated that the A regions of CNA, ACE, BA0871 and BA5258 adopt similar structural folds that are capable of accommodating a collagen triple helical structure, however, specific residues in the binding surfaces may determine the unique interactions between each MSCRAMM and collagen.

### EXAMPLE 13

BA0871 and BA5258 Mediate Bacterial Adherence to Collagen

To investigate if BA0871 and BA5258 are capable of mediating bacterial attachment to collagen, a surface display system was developed in a non-pathogenic heterologous host, S. carnosus strain TM300. The display vector pYX105 is capable of replicating in S. carnosus and contains DNA sequences for the promoter, signal peptide, the last B repeat and the cell wall anchoring region of the S. aureus collagen adhesin CNA. The A regions of BA0871 and BA5258 were cloned into pYX105 between the signal peptide and the B repeat sequence, allowing the surface display of the A regions in S. carnosus. The constructs then were electroporated into TM300 and the resulting strains were designated TM300(BA0871A) and TM300(BA5258A).

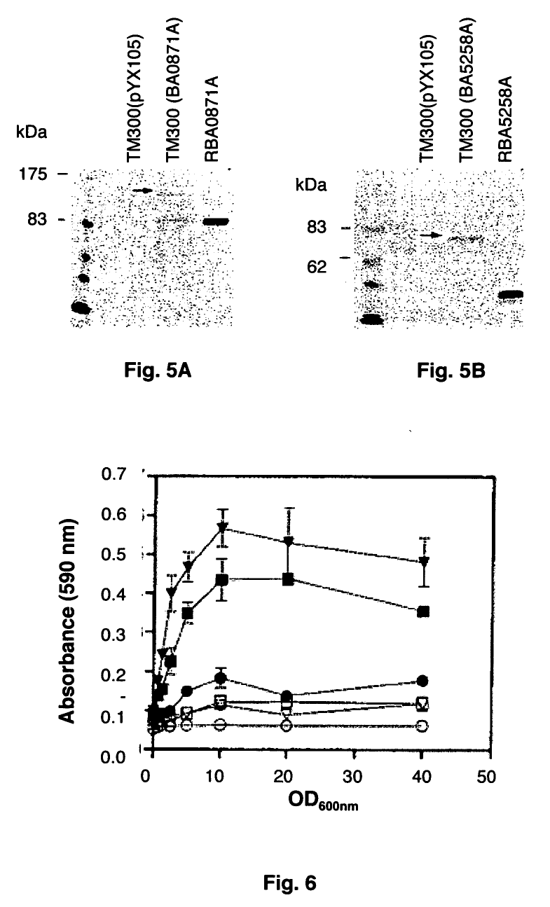

The gene products are fusions proteins containing the signal peptide of CNA, the A region of BA0871 or BA5258, respectively, the B repeat and the cell wall anchoring region of CNA. After post-translational processing by signal peptidase and sortase, the mature products, BA0871f and BA5258f, respectively should consist of the A region of BA0871 or BA5258, and the CNA B repeat as well as the first four residues of the LPXTG motif. The expected molecular sizes are 106930.1 Da for BA0871f and 65284.2 Da for BA5258f. The surface expression of the two A regions was verified by western blot analysis of lysostaphin cell wall extracts of the two strains. Bands of the expected sizes were observed and were indicated by arrows (FIGS. 5A-5B). In TM300(BA0871A), a smaller band that migrated at a similar rate as the recombinant A region of BA0871 was also observed and may be due to a proteolytic cleavage at the junction between the BA0871 A region and the CNA B repeat in the fusion protein.

To test if these recombinant strains are capable of adhering to immobilized collagen, cell attachment assays were carried out (FIG. 6). Exponential phase cells of TM300(BA0871A) and TM300(BA5258A) as well as TM300 containing vector pYX105 only were incubated with immobilized bovine type I collagen or BSA. The results showed that the expression of BA0871A and BA5258A on the surface of S. carnosus increased the ability of the bacteria to adhere to collagen by ˜3-4 fold, whereas their abilities to adhere to BSA remained at the low basal level. This suggests that the two proteins can act as collagen adhesins when displayed on bacterial surface.

The following references are cited herein.
1. Mock, M, and Fouet, A. (2001) Annu. Rev. Microbiol. Vol. 55, 647-671.
2. Hanna, P. (1998) Curr Top Microbiol Immunol. Vol. 225, 13-35.
3. Ascenzi, et al. (2002) FEBS Lett, Vol. 531, 384-388.
4. Patti et al. (1993) Biochemistry. Vol. 32, 11428-11435.
5. Patti et al. (1994) Infect. Immun. Vol. 62, 152-161.
6. El Tahir and Skurnik (2001) Int J Med Microbiol, Vol. 291, 209-218.
7. Pouttu et al. (1999) Mol Microbiol. Vol. 31, 1747-1757.
8. Rich et al. (1999) J. Biol. Chem. Vol. 274, 26939-26945.
9. Nallapareddy et al. (2003) Mol Microbiol, Vol. 47, 1733-1747.
10. Lannergard et al. (2003) FEMS Microb. Lett. Vol. 222, 69-74.
11. Shimoji et al. (2003) J Bacteriol, Vol. 185, 2739-2748.
12. Hienz et al. (1996) J. Infect. Dis. Vol. 174, 83-88.
13. Elasri etal. (2002) Bone 30, Vol. 275-280.
14. Rhem et al. (2000) Infect. Immun. Vol. 68, 3776-3779.
15. Mamo et al. (2000) Microbiol. Immunol. Vol. 44, 381-384.
16. Visai et al. (2000) J. Biol. Chem. Vol. 275, 39837-39845.
17. Xu et al. (2004) J. Infect. Dis. Vol. 189, 2323-2333.
18. Gripenberg-Lerche et al. (1994) Infect Immun Vol. 62, 5568-5575.
19. Gripenberg-Lerche et al. (1995) Infect Immun Vol. 63, 3222-3226.
20. Nilsson et al. (1998) J. Clin. Invest. Vol. 101, 2640-2649.
21. Symersky et al. (1997) Nat. Struct. Biol. Vol. 4, 833-838.
22. Deivanayagam et al. (2002) Embo, J, Vol. 21, 6660-6672.
23. Ponnuraj et al. (2003) Cell, Vol. 115, 217-228.
24. Sillanpaa et al. (July 2004) Microbiology, Vol. 150(Pt 7):2069-78.
25. Xu et al. (2000) J. Biol. Chem. Vol. 275, 38981-38989.
26. Yang et al. (1986) Methods Enzymol, Vol. 130, 208-269.
27. Provencher, S. W., and Glockner, J. (1981) Biochemistry, Vol. 20, 33-37.
28. Andrade et al. (1993) Protein Eng, Vol. 6, 383-390.
29. Sreerama, N, and Woody, R W (1993) Anal Biochem, Vol. 209, 32-44.
30. Johnson, W (1988) Annu Rev Biophys Biophys Chem, Vol. 17, 145-166.
31. Xu et al. (2002) J Biol Chem Vol. 277, 27312-27318.
32. Lee, C Y and Buranen, S L (1989) J. Bacteriol. Vol. 171, 1652-1657.
33. Eidhin et al. (1998) Mol. Microbiol. Vol. 30, 245-257.
34. Deivanayagam et al. (2000) Structure Fold Des. Vol. 8, 67-78.
35. Ponnuraj et al. (2002) Biochim Biophys Acta, Vol. 1596, 173-176.
36. Guidi-Rontani, C. (2002) Trends in Microbiology, Vol. 10, 405-409.
37. Hanna, P, and Ireland, J A (1999) Trends Microbiol, Vol. 7, 180-182.
38. Lacy and Collier, (2002) Curr Top Microbiol Immunol, Vol. 271, 61-85.
39. Ruthel et al. (2004) J Infect Dis, 189, 1313-1316.
40. Rich et al. (1999) J. Biol. Chem. Vol. 274, 24906-24913.
41. Bourgogne et al. (2003) Infect Immun, Vol. 71, 2736-2743.

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A DNA encoding a cell wall anchored protein of B. anthracis, comprising:
(a) isolated DNA which encodes a cell wall anchored protein of Bacillus anthracis;
(b) isolated DNA which hybridizes under high stringency conditions to the isolated DNA of (a) above and which encodes a cell wall anchored protein of Bacillus anthracis; or
(c) isolated DNA differing from the isolated DNAs of (a) or (b) above in codon sequence due to the degeneracy of the genetic code and which encodes a cell wall anchored protein.

2. The isolated DNA of claim 1, wherein said DNA has the nucleotide sequence shown in SEQ ID NO: 10 or SEQ ID NO: 11.

3. The isolated DNA of claim 2, wherein said DNA encodes a cell wall anchored protein having the amino acid sequence shown in SEQ ID NO: 12 or SEQ ID NO: 13.

4. The isolated DNA of claim 3, wherein said cell wall anchored protein has a collagen-binding region having a sequence shown in SEQ ID NO: 17 or SEQ ID NO: 18.

5. The isolated DNA of claim 3, wherein said DNA has a sequence that is about 90% homologous to the nucleotide sequence shown in SEQ ID NO: 10 or SEQ ID NO: 11.

6. A vector comprising the isolated DNA of claim 1 and regulatory elements necessary for expression of said DNA on the surface of a bacterium.

7. A host bacterium comprising and expressing the vector of claim 6.

8. A pharmaceutical composition comprising the isolated DNA of claim 1 and a pharmaceutically acceptable carrier.

9. An immunogenic composition comprising an immunogenically effective amount of the DNA of claim 1 and a pharmaceutically acceptable carrier, adjuvant or diluent or a combination thereof.

10. A method of inducing a host-mediated immune response against Bacillus anthracis in a subject, comprising:
administering the immunogenic composition of claim 9 to the subject, wherein the cell wall anchored protein expressed by the DNA comprising said immunogenic composition is effective to activate host immune cells against the protein such that subsequent presentation of said protein by Bacillus anthacis in the subject induces the host-mediated immune response against Bacillus anthracis.

11. The method of claim 10, wherein said DNA comprises a vector effective to express said DNA.

12. An isolated and purified cell wall anchored protein of B. anthracis encoded by the DNA of claim 1.

13. The isolated and purified cell wall anchored protein of claim 1, wherein said protein has a sequence shown in SEQ ID NO: 12 or SEQ ID NO: 13.

14. The isolated and purified cell wall anchored protein of claim 13, said protein comprising a collagen-binding region having a sequence shown in SEQ ID NO: 17 or SEQ ID NO: 18.

15. The isolated and purified cell wall anchored protein of claim 13, wherein said protein has a sequence that is about 90% homologous to a sequence shown in SEQ ID NO: 12 or SEQ ID NO: 13.

16. A pharmaceutical composition comprising the isolated and purified cell wall anchored protein of claim 12 and a pharmaceutically acceptable carrier.

17. An immunogenic composition comprising an immunogenically effective amount of the isolated and purified cell wall anchored protein of claim 12 and a pharmaceutically acceptable carrier, adjuvant or diluent or a combination thereof.

18. A method of inducing a host-mediated immune response against Bacillus anthracis in a subject, comprising:
administering the immunogenic composition of claim 17 to the subject, wherein the cell wall anchored protein comprising said immunogenic composition is effective to activate host immune cells against itself such that subsequent presentation of said protein by Bacillus anthracis in the subject induces the host-mediated immune response against Bacillus anthracis.

19. An isolated DNA encoding a collagen-binding region of a cell wall anchored protein of B. anthracis, comprising:
(a) isolated DNA which encodes a collagen-binding region of a cell wall anchored protein of Bacillus anthracis;
(b) isolated DNA which hybridizes under high stringency conditions to the isolated DNA of (a) above and which encodes a collagen-binding region of a cell wall anchored protein of Bacillus anthracis; or
(c) isolated DNA differing from the isolated DNAs of (a) or (b) above in codon sequence due to the degeneracy of the genetic code and which encodes a collagen-binding region of a cell wall anchored protein.

20. The isolated DNA of claim 19, wherein said DNA has the nucleotide sequence shown in SEQ ID NO: 15 or SEQ ID NO: 16.

21. The isolated DNA of claim 12, wherein said DNA has a sequence that is about 90% homologous to a sequence shown in SEQ ID NO: 15 or SEQ ID NO: 16.

22. A vector comprising the isolated DNA of claim 19 and regulatory elements necessary for expression of said DNA on the surface of a bacterium.

23. A host bacterium comprising and expressing the vector of claim 22.

24. A pharmaceutical composition comprising the isolated DNA of claim 19 and a pharmaceutically acceptable carrier.

25. An immunogenic composition comprising an immunogenically effective amount of the DNA of claim 19 and a pharmaceutically acceptable carrier, adjuvant or diluent or a combination thereof.

26. A method of inducing a host-mediated immune response against Bacillus anthracis in a subject, comprising:
administering the immunogenic composition of claim 25 to the subject, wherein the cell wall anchored protein expressed by the DNA comprising said immunogenic composition is effective to activate host immune cells against the protein such that subsequent presentation of said protein by Bacillus anthacis in the subject induces the host-mediated immune response against Bacillus anthracis.

27. The method of claim 26, wherein said DNA comprises a vector effective to express said DNA.

28. An isolated and purified collagen-binding peptide encoded by the DNA of claim 19.

29. The isolated and purified collagen-binding peptide of claim 28 having a sequence shown in SEQ ID NO: 17 or SEQ ID NO: 18.

30. The isolated and purified collagen-binding peptide of claim 21, wherein said peptide has a sequence that is about 90% homologous to a sequence shown in SEQ ID NO: 17 or SEQ ID NO: 18.

31. A pharmaceutical composition comprising the isolated and purified collagen-binding peptide of claim 28 and a pharmaceutically acceptable carrier.

32. An immunogenic composition comprising the isolated and purified collagen-binding peptide of claim 28 and a pharmaceutically acceptable carrier, adjuvant or diluent or a combination thereof.

33. A method of inducing a host-mediated immune response against Bacillus anthracis in a subject, comprising:
administering the immunogenic composition of claim 32 to the subject, wherein the collagen-binding peptide comprising said immunogenic composition is effective to activate host immune cells against itself such that subsequent presentation of said peptide by Bacillus anthacis in the subject induces the host-mediated immune response against Bacillus anthracis.
